(19) United States
(12) Patent Application Publication
(10) Pub. No.: US 2015/0037303 A1
(43) Pub. Date: Feb. 5, 2015

(54) CELLS, COMPOSITIONS, AND TREATMENT METHODS FOR STIMULATION OF HEMATOPOIESIS

(71) Applicant: Regen BioPharma, Inc, La Mesa, CA (US)

(72) Inventors: Thomas Ichim, San Diego, CA (US); David Koos, La Mesa, CA (US)

(73) Assignee: Regen BioPharma, Inc, La Mesa, CA (US)

(21) Appl. No.: 13/957,427

(22) Filed: Aug. 1, 2013

(51) Int. Cl. A61K 35/28; A61K 38/20; A61K 38/18; A61K 38/19; A61K 35/12; A61K 35/14

(52) U.S. Cl. CPC A61K 35/28; A61K 35/35; A61K 35/17; A61K 35/15; A61K 38/18; A61K 38/193; A61K 38/2006; A61K 38/204; A61K 38/2046; A61K 38/1858
USPC 424/93.71; 424/93.7

(57) ABSTRACT

The invention discloses novel methods, compositions of matter, and kits for the treatment of disorders affecting the hematopoietic system. Patients are administered an autologous cellular mixture derived from adipose stromal vascular fraction, said cellular mixture comprising endothelial cells, endothelial progenitor cells, T regulatory cells, monocytes, and hematopoietic stem cells. In one embodiment, treatment is provided for patients suffering from inflammatory disorders including aplastic anemia.

### CROSS-REFERENCE TO RELATED APPLICATIONS

This application claims priority to U.S. Provisional Application No. 61/670,791, filed Sep. 12, 2012, and entitled “Cells, Compositions, and Treatment Methods for Stimulation of Hematopoiesis”, which is hereby expressly incorporated by reference in its entirety.

### FIELD OF THE INVENTION

The invention relates to the field of stem cell biology, cell culture, hematology, and hematopoietic stimulation. In particular, the invention relates to the area of adjuvant therapies for hematopoietic reconstitution. More specifically, the invention discloses methods, means, and compositions of matter useful for the treatment of hematopoietic disorders through the use of autologous, non-expanded, endothelial cells and endothelial progenitor cells (EPC) derived from the stromal vascular fraction of patients with hematological conditions associated with inflammation.

### BACKGROUND

Endothelial cells have been previously shown in the art to stimulate hematopoietic reconstitution. Endothelial progenitor cells have been shown to possess various regenerative abilities. Autologous, adipose-derived, stromal vascular fraction possesses EPC and hematopoietic stem cells, as well as regulatory cells, that augment hematopoietic reconstitution while reducing the potential immune response.

### SUMMARY

Embodiments herein are directed to methods for repairing a hematopoietic defect in a patient, said method consisting of: a) selecting a patient in need of therapy; b) obtaining a population of autologous cells from adipose tissue; c) isolating the stromal vascular fraction; d) manipulating said adipose tissue to isolate and purify a cellular component; and e) infusing said cellular component into said patient.

### BRIEF DESCRIPTION OF THE DRAWINGS

FIG. 1 is a graph showing Human SVF induces acceleration of Leukocyte Rebound Post 5-FU Insult

### DETAILED DESCRIPTION

The invention teaches the use of stromal vascular fraction (SVF) in the treatment of hematological diseases. In one embodiment, the invention provides methods of treating patients with hematological diseases associated with inflammation, including aplastic anemia, immune thrombocytopenic purpura, graft versus host disease, chemotherapy induced bone marrow damage, and radiation induced bone marrow aplasia.

An SVF preparation is generated using autologous adipose tissue. Numerous means of extracting adipose tissue are known in the art. In one particular example, the technique clinically used for SVF autologous treatment of rheumatoid arthritis (Rodriquez et al International Archives of Medicine 2012, 5:5) is utilized. Specifically, SVF cells are isolated and prepared under the guidelines of Good Tissue Practices 21 CFR 1271 as relates to sample screening and processing in the sterile flow hood, inside of a class 10000 clean room. SVF cells are isolated by first washing 500 cc of lipoaspirate with PBS and subsequently, the cells are transferred to 175 ml sterile centrifuge containers followed by the addition of collagenase solution for a final concentration of 0.048%. The centrifuge containers are subsequently sealed and placed in an elliptical shaker and incubated at 37 C for 60-80 minutes. The content of the tubes are then filtered through a cell strainer into sterile 50 ml centrifuge tubes and centrifuged for 12 min at 800 rcf. During centrifugation, SVF cells are concentrated to the bottom of the container while the adipocyte layer and debris remained suspended. Following centrifugation, the stromal cells are then resuspended in 5 mL of autologous serum for enzyme inactivation then washed 2 times with PBS.

All the cells are aliquoted in cryovials, frozen in liquid nitrogen and stored until use. When the patient is prepared to receive cells, the cells are assessed for viability, endotoxin, and contamination before treatment is performed. In one embodiment, the patient is allowed to heal from the liposuction for one week. Alternative times may be utilized within the practice of the invention. The scope of the delay between extraction and infusion is so that infused cells will not all home to the tissue injury caused by the liposuction. For each treatment session, the cells are rinsed with PBS and Human AB serum after thawing, diluted in saline solution and autologous serum, loaded into sterile syringes, and then transported in a controlled temperature cooler accompanied by the corresponding certificate and delivered to the physician for infusion.

Patients receive intravenous injection (2×106 cells per ml diluted in saline solution). Multiple injections of cells may be administered at various time points to modify therapeutic effects. Frequency of administration may be dependent on inflammatory, hematological, and immunological markers.

It is known that patients with aplastic anemia have a deficiency in numbers of T regulatory cells, as well as enhanced activity of Th17 cells. In one study, Kordasti et al. investigated 63 patients with acquired AA. Th1 and Th2 cells were significantly higher in AA patients than in healthy donors. Tregs were significantly lower in patients with severe AA than in healthy donors and patients with non-severe AA. Th17 cells were increased in severe AA but normal in non-severe AA. Activated and resting Tregs were reduced in AA, whereas cytokine-secreting non-Tregs were increased. Tregs from AA patients were unable to suppress normal effector T cells. In contrast, AA effector T cells were suppressible by Tregs from healthy donors. Th1 clonality in AA, investigated by high-throughput sequencing, was greater than in healthy donors. Our results confirm that Th1 and Th2 cells are expanded and Tregs are functionally abnormal in AA.

The clonally restricted expansion of Th1 cells is most likely to be antigen-driven, and induces an inflammatory environment, that exacerbate the functional impairment of Tregs, which are reduced in number. This suggests that one of the effector processes in AA is the immunologically-mediated bone marrow damage, which is in part associated with lack of T regulatory cell number and activity. The stromal vascular fraction contains high numbers of T regulatory cells, as well as in addition to anti-inflammatory monocytes, which were described above, also mesenchymal stem cells , which are capable of inducing generation of Treg, as well as directly inhibiting Th17 cells. Thus in one embodiment of the invention, the T regulatory cell content of the SVF is utilized to modulate immunity.

The method may also be applied to the treatment of graft versus host disease for bone marrow and cord blood transplants. In which case, human adipose tissues are obtained by simple liposuction from the abdominal subcutaneous fat abdominoplasty patients. Subcutaneous adipose tissues are digested with collagenase I (1 mg/ml) under gentle agitation for 60 min at 37° C. The digested tissues are centrifuged and the pellet (stromal vascular fraction is resuspended in Dulbecco's modified Eagle's medium (DMEM, Invitrogen, Carlsbad, Calif., USA) media containing 0.2 mmol/L ascorbic acid and 10% fetal bovine serum (FBS) obtained from bovine spongiform encephalopathy free herd. The cell fraction was cultured overnight at 37° C./5% CO2 in DMEM-based media containing 0.2 mmol/L ascorbic acid and 10% FBS. After 24 h, the cell medium was changed to Keratinocyte-SFM (Invitrogen)-based media containing 0.2 mmol/L ascorbic acid, 0.09 mmol/L calcium, 5 ng/ml rEGF, and 5% FBS. The cells were subculture expanded in the same media until passage 3. Characteristics of ATMSC including surface marker expression and angiogenic factor secretion were examined in our previous studies.11,15,16 FBS contaminant from cultured MSC were completely removed by several washing with PBS and was verified through the test of albumin concentration below the measurement limit using a bovine albumin enzyme-linked immunosorbent assay quantitiation kit (Bethyl Laboratories, Montgomery, Tex., USA). Aliquots of the ATMSC are then tested for cell viability and fungal, bacterial, endotoxin, and mycoplasma contamination as demanded by the Code of Federal Regulations, Title 21 (21CFR) before further use. In vitro hypoxia experiments are performed with a hypoxic incubator (APM-30D, ASTEC, Japan) that continuously infuses a calibrated gas mixture (95% N2, 5% CO2). Experiments were performed at oxygen concentrations of 21% and 1%.

### EXAMPLES

### Example 1

### Prophetic Example—Pilot Trial in Treatment of Aplastic Anemia

An open label trial is performed in 10 patients with aplastic anemia. Patients will have specific inclusion and exclusion criteria.

### Inclusion Criteria:

Diagnosis of Aplastic Anemia (AA).

There must be at least two of the following: hemoglobin <100 g/L; platelet count <50×109/L; neutrophil count <1.5×109/L, and a hypocellular bone marrow.

AA as defined by a hypocellular bone marrow of <25% cellularity and two of the following: neutrophil count <0.5×109/L platelets <20×109/L reticulocytes <20×109/L AA as defined by a hypocellular bone marrow and cytopenia in at least two cell lines and neutrophil count >0.5×109/L, and red cell and/or platelet transfusion dependence.

Patients with a history of AA must have had an incomplete response at least 3 months following treatment with ATG/CsA, or they must have relapsed following an initial response to treatment, and they do not have a HLA-matched donor for bone marrow transplantation. Patients with a history of AA must have red cell and/or platelet transfusion dependence.

Peripheral blood counts at the time of enrollment must include at least one of the following: haemoglobin <90 g/L or red blood cell (RBC) transfusion dependence, PMN <1×109/L, or platelet count <50×109/L.

Patients must have organ function defined as follows below: total bilirubin within normal institutional limits (NV: 0.0-20.5 umol/L) AST(SGOT)/ALT(SGPT) <2.5× institutional upper limit of normal AST (NV: 0-35 U/L); ALT (NV: 0-40 U/L) creatinine within normal institutional limits (NV: 53-106 umol/L) or creatinine clearance >1.25 ml/s for patients with creatinine levels above institutional normal.

Relapse/refractory to at least 1 immunosuppressive first line treatment.

Not eligible for allogeneic bone marrow transplantation.

### Exclusion Criteria:

Previous or current malignancy.

Active or latent infectious disease.

Positive serologic tests for HIV, HCV, HBV, HTLV-1 and 2, Syphilis or Chagas disease.

Previous drug reaction for antithymocyte globulin, cyclosporin or corticosteroids.

Severe organic impairment (renal, hepatic, cardiac, pulmonary).

Uncontrolled hypertension or diabetes.

Pregnancy.

Previous history of allergic reaction to penicillin or streptomycin.

Severe psychiatric disorder.

After standard immunosuppressive therapy with rabbit antithymocyte globulin 3.5 mg/Kg/day during 5 days, autologous SVF is administered at day 0, 14, and 28. Oral cyclosporine 5 mg/Kg/day (with dose correction weekly to keep serum cyclosporine level between 150-250 mg/dl) up to 6 months is added.

SVF cells are collected from 500 cc of adipose tissue for each patient and frozen until use. SVF cells are prepared according to the method of Tzouvelekis (Tzouvelekis et al., J Transl Med. 2011 Oct. 21; 9:182). Eligible patients undergo lipoaspiration under general anesthesia in a sterile surgical operating room setting. Approximately 100-500 gr of adipose tissue is isolated from the above procedure performed by a plastic surgeon. Enzyme dissolution procedure of the adipose tissue, using collagenase type I solution under agitation for 2 hours plus 10 cc of lecithine followed by two centrifugations at 100 g for 10 minutes the first and at 1800 g for 10 minutes the second, is performed to separate the stromal cell fraction (pellet) from adipocytes. The pellets are treated with red lysis buffer. The final pellet will be re-suspended and a small volume of suspension is used for flow cytometry analysis, counting CD29, CD73, CD90, CD34, CD105, positive cells and CD14, CD31 and CD45 negative cells in Coulter Epics XL/MCL. 10 mL of the suspension will be assessed for bacterial check with the BacT/Alert system (with colorimetric carbon dioxide detector). In the case of an infected sample the microorganism is identified with VITEK 2 Compact 15 and excluded. A volume of DMSO solution is added in the remaining suspension so that the final volume contains 10% DMSO and 2% Haes-steril 200. The cells are cryo-preserved gradually and stored in cryovials, air-tightly sealed with cryoflex membrane. The vials are then placed in constant temperature in liquid nitrogen and will be stored there. Viability of the cells is estimated by tryphan blue. A total of 3-5×106 cells per gram of adipose tissue is expected to be isolated.

Based on the current literature (Mitchell et al. Stem Cells 2006, 24:376-385), since SVF represents an heterogeneous cell population it is anticipated that approximately 50-70% of the total number of isolated cells to be of mesenchymal origin meaning CD29, CD105, CD90, CD73 positive and CD34, CD45 negative cells and 20-30% mature endothelial (CD31 and positive) and hematopoetic (CD34 positive) cells.

The clinical endpoint shall be assessed six months after treatment. A successful trial result includes no major treatment associated adverse events, particularly no allergic reactions, infectious diseases, organ dysfunction or other related to the SVF infusion. Additionally, there should be a reduction in the level of cytopenias at six months post treatment, as well as reduced transfusional requirements in terms of units of blood or platelets transfused after the SVF infusion. Furthermore, there should be a demonstrated decrease in the incidence of infections and febrile neutropenia

### Example 2

### Working Example Pilot Animal Study Evaluating Effects of Human SVF of Murine Endogenous Hematopoietic Reconstitution

Purpose: To determine whether intravenous administration of human stromal vascular fraction augments endogenous hematopoietic activity in a murine model of hematopoietic injury. Study Limitation: The administration of human SVF cells into mice may not detect species-specific growth factors produced by the human cells. Additionally, a concern exists regarding xenogenic immunity.

Detailed Description of Study Design: 8-12 week female B6 mice were treated with intraperitoneal injection of 150 mg/kg 5-FU (Sigma Chemical Corp., St. Louis, Mo.) on day 0 to induce a state of myelosuppression mimicking aplastic anemia. Groups of 10 mice each were administered phosphate buffered saline (PBS) control or 15,000; 30,000; or 60,000 SVF generated according to the CMC section of the IND from healthy adult donors on day 1 after administration of 5-FU. Survival and complete blood counts were evaluated over a period of 2 weeks. Blood samples (20 uL) were taken retro-orbitally in a vial containing 1 uL of 0.5 M EDTA and cell counts analyzed using a Coulter Onyx. Blood analysis was performed every 2 days.

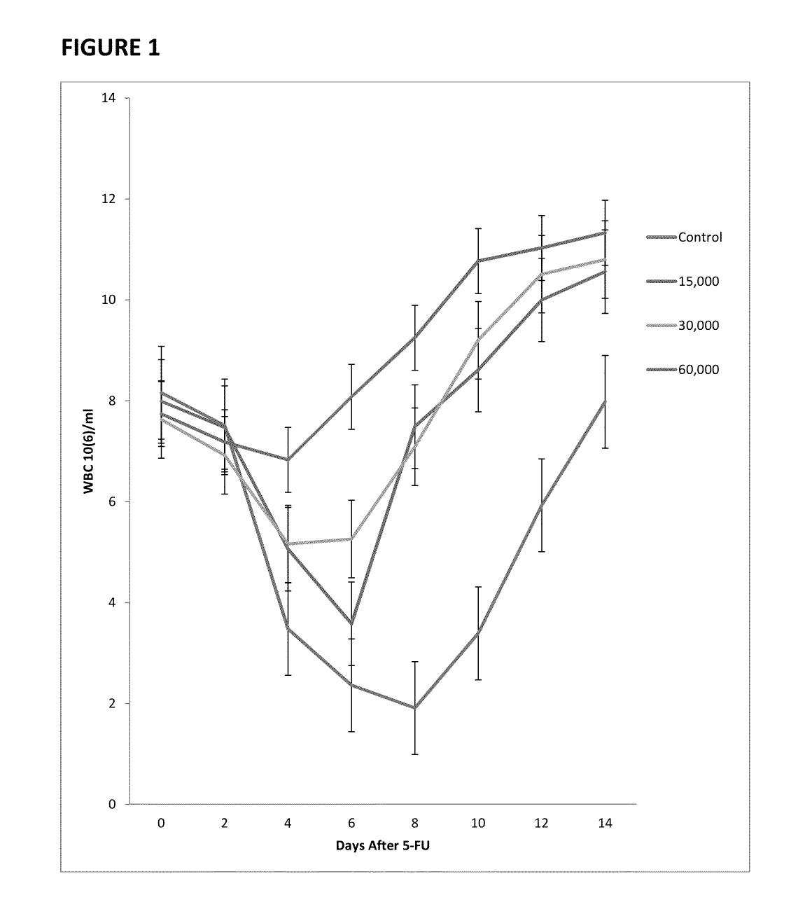

Study Results: Animals were examined every second day for the duration of the experiment for signs of distress, including mobility, hair ruffling, and general appearance. No adverse events were visible on general examination in any of the injected groups. An increase in reconstitution of WBC was observed in animals treated with human SVF cells after administration of 5-FU as compared to controls that were administered PBS only (FIG. 1 and Table 1).

TABLE 1

| | Days | | | | | | | |
|---|---|---|---|---|---|---|---|---|
| | 0 | 2 | 4 | 6 | 8 | 10 | 12 | 14 |
| Control | 8.2 | 7.7 | 3.9 | 2.4 | 1.3 | 2.8 | 5.1 | 7.4 |
| | 8.9 | 6.8 | 3.3 | 2.3 | 1.7 | 3.1 | 7.3 | 10.6 |
| | 7.7 | 7 | 2.8 | 2.9 | 2.4 | 4.8 | 5.9 | 8.1 |
| | 7.5 | 7.9 | 3.7 | 1.7 | 1.6 | 4.1 | 6.4 | 8.1 |
| | 8.7 | 7.4 | 4.7 | 3.5 | 2.2 | 3.1 | 6.3 | 7.1 |
| | 9 | 7.2 | 3.2 | 1.3 | 1.4 | 2.9 | 6.3 | 8.1 |
| | 7.3 | 6.9 | 2.8 | 2.3 | 1.4 | 4.6 | 7.1 | 8.1 |
| | 7.9 | 7.5 | 3.8 | 2.2 | 1.7 | 1.1 | 3.6 | 6.2 |
| | 8.2 | 8.5 | 4.1 | 2.7 | 2.2 | 3.1 | 4.2 | 7.2 |
| | 8.2 | 8.2 | 2.5 | 2.3 | 3.2 | 4.3 | 7.1 | 8.9 |
| Control | 8.16 | 7.51 | 3.48 | 2.36 | 1.91 | 3.39 | 5.93 | 7.98 |
| stdev | 0.577735 | 0.566569 | 0.682805 | 0.605897 | 0.591514 | 1.098939 | 1.257025 | 1.185842 |

Implications: The doses of 15,000; 30,000; and 60,000 SVF cells represent doses for a 70 kg human of 52,500,000; 105,000,000; and 210,000,000. These doses bracket the proposed clinical trial dose of 100 million cells per patient. We conclude the possibility that SVF may be safe and useful for the acceleration of hematopoietic reconstitution.

One skilled in the art will appreciate that these methods and devices are and can be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure.

It is apparent to one skilled in the art that varying substitutions and modifications can be made to the invention disclosed herein without departing from the scope and spirit of the invention. Furthermore, those skilled in the art recognize that the aspects and embodiments of the invention set forth herein can be practiced separate from each other or in conjunction with each other. Therefore, combinations of separate embodiments are within the scope of the invention as disclosed herein.

### BIBLIOGRAPHY

Astori, G., et al., “In vitro” and multicolor phenotypic characterization of cell subpopulations identified in fresh human adipose tissue stromal vascular fraction and in the derived mesenchymal stem cells. J Transl Med, 2007. 5: p. 55.

Bastard, J. P., et al., Recent advances in the relationship between obesity, inflammation, and insulin resistance. Eur Cytokine Netw, 2006. 17(1): p. 4-12.

Casteilla, L., et al., Adipose-derived stromal cells: Their identity and uses in clinical trials, an update. World J Stem Cells, 2011. 3(4): p. 25-33.

Cipolletta, D., et al., Tissular T(regs): a unique population of adipose-tissue-resident Foxp3+CD4+ T cells that impacts organismal metabolism. Semin Immunol, 2011. 23(6): p. 431-7.

Gustafsson, C., et al., Gene expression profiling of human decidual macrophages: evidence for immunosuppressive phenotype. PLoS ONE, 2008. 3(4): p. e2078.

Kordasti, S., et al., Functional characterization of CD4+ T cells in aplastic anemia. Blood, 2012. 119(9): p. 2033-43.

Mantovani, A., et al., Macrophage polarization: tumor-associated macrophages as a paradigm for polarized M2 mononuclear phagocytes. Trends Immunol, 2002. 23(11): p. 549-55.

Mehta, A., et al., Infection-induced modulation of m1 and m2 phenotypes in circulating monocytes: role in immune monitoring and early prognosis of sepsis. Shock, 2004. 22(5): p. 423-30.

Odegaard, J. I., et al., Macrophage-specific PPARgamma controls alternative activation and improves insulin resistance. Nature, 2007. 447(7148): p. 1116-20.

Pachon-Pena, G., et al., Stromal stem cells from adipose tissue and bone marrow of age-matched female donors display distinct immunophenotypic profiles. J Cell Physiol, 2011. 226(3): p. 843-51.

Ponomarev, E. D., et al., CNS-derived interleukin-4 is essential for the regulation of autoimmune inflammation and induces a state of alternative activation in microglial cells. J Neurosci, 2007. 27(40): p. 10714-21.

Song, G. Y., et al., Evolution of an immune suppressive macrophage phenotype as a product of P38 MAPK activation in polymicrobial sepsis. Shock, 2001. 15(1): p. 42-8.

Suganami, T., J. Nishida, and Y. Ogawa, A paracrine loop between adipocytes and macrophages aggravates inflammatory changes: role of free fatty acids and tumor necrosis factor alpha. Arterioscler Thromb Vasc Biol, 2005. 25(10): p. 2062-8.

Wang, Y., et al., Ex vivo programmed macrophages ameliorate experimental chronic inflammatory renal disease. Kidney Int, 2007. 72(3): p. 290-9.

Zeyda, M. and T. M. Stulnig, Adipose tissue macrophages. Immunol Lett, 2007. 112(2): p. 61-7.

Zeyda, M., et al., Human adipose tissue macrophages are of an anti-inflammatory phenotype but capable of excessive pro-inflammatory mediator production. Int J Obes (Lond), 2007. 31(9): p. 1420-8.

1. A method for repairing a hematopoietic defect in a patient, said method consisting of: a) selecting a patient in need of therapy; b) obtaining a population of autologous cells from adipose tissue; c) isolating the stromal vascular fraction; d) manipulating said adipose tissue to isolate and purify a cellular component ; and e) infusing said cellular component into said patient.

2. The method of claim 1, wherein said disorder of the hematopoietic system is associated with inflammation selected from a group comprising of: a) immune thrombocytopenic purpura; b) aplastic anemia; c) graft versus host disease; d) chemotherapy induced bone marrow damage; e) radiation induced bone marrow aplasia; f) bone marrow failure; g) bone marrow transplantation; and h) cord blood transplantation.

3. The method of claim 1, wherein said cells are isolated from adipose stromal vascular fraction.

4. The method of claim 1, wherein said cells isolated from adipose tissue are endothelial cells.

5. The method of claim 1, wherein said cells isolated from adipose tissue are endothelial progenitor cells.

6. The method of claim 1, wherein said cells isolated from adipose tissue are T regulatory cells.

7. The method of claim 1, wherein said cells isolated from adipose tissue are Type 2 monocytes.

8. The method of claim 1, wherein said cells isolated from adipose tissue are hematopoietic stem cells.

9. The method of claim 1, wherein said purified cellular component is concentrated for immune modulatory cells.

10. The method of claim 1, wherein said purified cellular component is concentrated for angiogenesis stimulating cells.

11. The method of claim 1, wherein said cell isolated from adipose stromal vascular fraction expresses a marker selected from a group of markers comprising: CD29, CD44, CD71, CD90, CD105/SH2, and/or SH3.

12. The method of claim 1, wherein said cells isolated by enzymatic digestion of adipose tissue substantially lack expression of a marker selected from a group of markers comprising: CD31, CD34, and/or CD45.

13. The method of claim 1, wherein said purification of cellular component is performed using a method selected from a group comprising of: a) enzymatic digestion; b) mechanical dissociation; c) ultrasound dissociation; and d) laser dissociation.

14. The method of claim 12, wherein said enzymatic digestion is performed by exposure of said adipose tissue to an enzyme selected from a group comprising of trypsin, collagenase, and dispase.

15. The method of claim 1, wherein said cellular component is administered intravenously prior to, at the moment of, or subsequent to exposure to an agent or plurality of agents causing impairment of hematopoietic tissue.

16. The method of claim 1, wherein said composition is administered together with a growth factor capable of stimulating proliferation and/or differentiation of hematopoietic stem cells.

17. The method of claim 16, wherein said growth factor is selected from a group of growth factors comprising of: a) G-CSF; b) M-CSF); c) GM-CS; d) stem cell factor; e) IL-1; f) IL-6; g) thrombopoietin; h) IL-7; and i) PDGF.

18. The method of claim 1, wherein said adipose-derived, stromal vascular fraction cells are autologous to the recipient in need of hematopoietic reconstitution.

19. A method of treating a disorder of the hematopoietic system associated with inflammation wherein said method of claim 1 provides for immune modulation associated with inflammation.
